(19) United States
(12) Patent Application Publication
(10) Pub. No.: US 2024/0424293 A1
(43) Pub. Date: Dec. 26, 2024

(54) WIRELESS IONTOPHORESIS DEVICE

(71) Applicants: Robert Worobey; Joshua Brown, Pittsburgh, PA (US); Garrett Grindle, Pittsburgh, PA (US); Anton Sabiev, Bronx, NY (US); William Bauman, Bronx, NY (US); Mark Korsten, Bronx, NY (US)

(72) Inventors: Robert Worobey; Joshua Brown, Pittsburgh, PA (US); Garrett Grindle, Pittsburgh, PA (US); Anton Sabiev, Bronx, NY (US); William Bauman, Bronx, NY (US); Mark Korsten, Bronx, NY (US)

(21) Appl. No.: 18/690,053

(22) PCT Filed: Sep. 6, 2022
(86) PCT No.: PCT/US2022/042600

§ 371 Date: Mar. 7, 2024

Related U.S. Application Data
(60) Provisional application No. 63/241,151, filed on Sep. 7, 2021.
(60) Provisional application No. 63/320,762, filed on Mar. 17, 2022.

(51) Int. Cl. A61N 1/30; A61N 1/02; A61N 1/04

(52) U.S. Cl. CPC A61N 1/303; A61N 1/025; A61N 1/0444; A61N 1/0448

(57) ABSTRACT

An iontophoresis device (10) includes a membrane applied to said skin having a drug delivery aperture, a solvent delivery aperture and a vent aperture for applying a vacuum between the membrane and skin. A drug cartridge defines a drug containing chamber in communication with the drug delivery aperture and a drug air chamber separated by a first flexible membrane. A solvent cartridge defines a solvent containing chamber and a solvent air chamber separated by a second flexible membrane. An electrode is positioned between the membrane and the skin and surrounds the drug delivery aperture and the solvent delivery aperture. A drug cartridge may also include two separate chambers, one containing a lyophilized agent, the other containing a solvent. The solvent and the agent remain separate until mixed during operation of the device.

### CROSS REFERENCE TO RELATED APPLICATIONS

This application is based upon and claims benefit of priority to U.S. Provisional Application No. 63/241,151, filed Sep. 7, 2021 and U.S. Provisional Application No. 63/320,762, filed Mar. 17, 2022, both of which are hereby incorporated by reference herein.

### FIELD OF THE INVENTION

This disclosure relates to an iontophoresis device for delivering medicine through a patient's skin. In more particular, this disclosure relates portable iontophoresis device that allows self-administration in a convenient and reliable fashion.

### BACKGROUND

Iontophoresis (ION) is a technique by which a low electric current is establish an electric field to drive ions across the skin. This technique has been used to deliver a number of classes of drugs, but predominantly for the treatment of local tissue injury and not for therapeutic systemic effect. In addition, iontophoresis devices are commonly large wired devices requiring treatment to be implemented at medical facilities rather than allowing self-administered treatment in a residential or other non-medically supervised facility.

In one particular non-limiting example, constipation and straining during bowel movement, collectively referred to as “difficulty with evacuation” (DWE), are common complaints in clinical settings and are often resistant to the usual interventions in persons with neurogenic bowel disorders. Fecal incontinence (FI) is reported to occur in approximately 10% of patients with chronic constipation, but it is suspected to occur at higher rates, being underreported due to patient embarrassment. Neostigmine (NEO) is known to have an effect on gastrointestinal (GI) function and is useful to induce a prompt and predictable bowel movement. NEO is a cholinergic agent which, when concomitantly administered with glycopyrrolate (GLY), a cardiopulmonary selective anticholinergic, has been shown to be safe and effective in promoting bowel evacuation and well tolerated in persons with spinal cord injury (SCI). The observed effectiveness and reliability of the combination of these agents is far greater than that of oral or rectal cathartics. The aforementioned combination of these medications has shown to be effective when delivered by intravenous or intramuscular routes. Neither intravenous nor intramuscular administration of NEO and GLY, however, are suitable for the practical, long-term bowel care in people with chronic constipation. It would, therefore, be beneficial to have a reliable and safe method to induce a prompt bowel evacuation that is suitable for long-term outpatient use.

In summary a wireless ION device to systemically deliver a positively charged compound in a safe manner would be highly desirable. A wireless ION device capable of delivering any number of other positively charged agents would expand the range of self-administered pharmacokinetics. The ability to use a wireless ION device is far more practical for patients to use, especially those with SCI, which will permit the self-administration of these agents in the home setting to induce a bowel evacuation. This novel approach for the delivery of these agents to induce a safe and predictable bowel evacuation holds the potential to dramatically improve bowel care for individuals with SCI.

### SUMMARY

This disclosure concerns an iontophoresis device for delivering a drug to a patient across a layer of skin. In one aspect, the iontophoresis device includes a membrane applied to the skin, the membrane having a drug delivery aperture, a solvent delivery aperture and a vent aperture for applying a vacuum between the membrane and the skin. A drug cartridge defines a drug containing chamber and a drug air chamber, the drug containing chamber and the drug air chamber separated by a first flexible membrane. The drug containing chamber is in fluid communication with the drug delivery aperture. The drug cartridge has a first drug cartridge port in fluid communication with the drug air chamber for pressurizing the drug containing chamber and a second drug cartridge port in fluid communication with the drug delivery aperture. The device further includes a solvent cartridge defining a solvent containing chamber and a solvent air chamber. The solvent containing chamber and the solvent air chamber are separated by a second flexible membrane. The solvent containing chamber is in fluid communication with the solvent vehicle aperture. The solvent cartridge has a first solvent port in fluid communication with the solvent air chamber for pressurizing the solvent containing chamber and a second solvent port in fluid communication with the solvent delivery aperture. An electrode extends between a positive terminal and a negative terminal. The electrode is positioned between the membrane and the skin and surrounds the drug delivery aperture and the solvent delivery aperture.

In another aspect of the disclosure, a removable delivery apparatus including a compressor is in communication with a processor configured with logic controller to control the compressor to deliver a vacuum to the vent aperture and a pressure to said first drug cartridge port and said first solvent cartridge port. The logic controller is further configured to supply current to the electrode. The drug cartridge and the solvent cartridge are housed within the removable delivery apparatus.

In another aspect of the disclosure, a non-permeable frame is in communication with the membrane. The non-permeable frame defines a drug receiving chamber and a solvent receiving chamber. The drug receiving chamber is in communication with the drug delivery aperture and the solvent receiving chamber is in communication with the solvent receiving aperture. The electrode is positioned between said non-permeable frame and the skin.

In another aspect of the disclosure, a semi-permeable membrane is in communication with the drug delivery aperture, the solvent delivery aperture and the vent aperture. The semi-permeable membrane transferring vacuum from the vent aperture to the drug delivery aperture and to the solvent delivery aperture.

In another aspect of the disclosure, the non-permeable frame and the semi-permeable membrane are positioned on opposing surfaces of the membrane. The iontophoresis device further includes a non-permeable cover configured to surround the semi-permeable membrane. The non-permeable cover is configured to engage the removable delivery apparatus.

In another aspect of the disclosure, the semi-permeable membrane is positioned between the non-permeable frame and the membrane. The iontophoresis device further includes a bleeder cloth positioned between the semi-permeable membrane and the membrane. The bleeder cloth is configured to distribute vacuum between the vent aperture and the drug delivery aperture and the solvent delivery aperture.

In another aspect of the disclosure, an electrode film layer is positioned between the semi-permeable membrane and non-permeable frame, the electrode film layer transferring current from the removable delivery apparatus to the electrode.

In another aspect of the disclosure, a conductive drug nozzle is in communication with the electrode film layer, the drug delivery aperture, and the drug receiving chamber, the conductive drug nozzle transferring current from the removable delivery apparatus to the electrode film layer. A conductive solvent nozzle is in communication with the electrode film layer, the solvent deliver aperture, and the solvent receiving chamber. The conductive solvent nozzle transferring current from the electrode film layer to the removable delivery apparatus.

In another aspect of the disclosure, the drug cartridge and the solvent cartridge comprise removable and refillable cartridges. A first conductive spring pin engages a first conductive portion of the drug cartridge. The first conductive spring pin retains the drug cartridge within the removable delivery apparatus and transfers current from the removable delivery apparatus the said first conductive portion to the conductive drug nozzle. A second conductive spring pin engages a second conductive portion of the solvent cartridge. The second conductive spring pin retains the solvent cartridge within the removable delivery apparatus and transferring current from the conductive solvent nozzle through the second conductive portion to the removable delivery apparatus.

The invention further encompasses an iontophoresis device characterized by a membrane applied to the skin, the membrane having a barb projecting therefrom and defining a bore providing fluid communication through the membrane to the skin. Intake openings in the barb provide fluid communication with the bore, and a vent aperture allows application of a vacuum between the membrane and the skin in this example embodiment.

A drug cartridge defines a solvent chamber and an agent chamber adapted to contain a lyophilized agent. The solvent chamber and the agent chamber are separated by an impermeable membrane. The agent chamber is in fluid communication with the bore through the intake openings. The solvent chamber is in fluid communication with a compressor for pressurizing the solvent chamber.

A barb isolation chamber is positioned within the agent chamber. The barb isolation chamber is adapted to receive the barb. A self-sealing membrane isolates the barb isolation chamber from the agent chamber. A plurality of cartridge openings are positioned in the barb isolation chamber to provide fluid communication between the agent chamber and the barb isolation chamber.

When the barb is received within the barb isolation chamber the barb pierces the self-sealing membrane and the impermeable membrane thereby releasing solvent from the solvent chamber into the agent chamber. The solvent mixes with the agent and enters the bore through the cartridge openings and the intake openings for application to the skin.

By way of example, the iontophoresis device may be further characterized by

a removable delivery apparatus including a compressor in communication with a processor configured with logic controller to control the compressor to deliver a vacuum to the vent aperture and a pressure to the solvent chamber.

In an example embodiment, a plurality of turbulent flow extrusions may be positioned within the agent chamber. The turbulent flow extrusions are adapted to create turbulent flow of the solvent and thereby promote mixing of the solvent and the agent.

Further by way of example, the self-sealing membrane seals around the barb to force solvent to flow through the cartridge openings when the barb pierces the self-sealing membrane. In an example embodiment the barb comprises a pointed tip and may further comprise a plurality of shaped cavities arranged proximate to the barb tip. The shaped cavities are adapted to allow free flow of the solvent between the solvent chamber and the agent chamber when the barb pierces the impermeable membrane. In an example embodiment the impermeable membrane may comprise a thin foil separation membrane. Further by way of example, a flexible membrane may be positioned within the solvent chamber.

An example iontophoresis device according to the invention may further include cartridges comprising a solvent in the solvent chamber and an agent in the agent chamber. In a particular example, the solvent comprises distilled water and the agent comprises neostigmine and glycopyrrolate.

### BRIEF DESCRIPTION OF THE DRAWINGS

FIG. 1 is a cross-sectional view of an example iontophoresis device in accordance with one aspect of the current disclosure;

FIG. 2 is an isometric top view of a patient application assembly for use in the iontophoresis device shown in FIG. 1;

FIG. 3 is an isometric bottom view of the patient application assembly shown in FIG. 2;

FIG. 4 is an isometric view of a non-permeable cover for use with the iontophoresis device shown in FIG. 1;

FIG. 5 is a cross-sectional view of the patient application assembly shown in FIGS. 2 and 3;

FIG. 6 is a cross-sectional view of an example iontophoresis device in accordance with another aspect of the current disclosure;

FIG. 7 is an isometric top view of a patient application assembly for use in the iontophoresis assembly shown in FIG. 6;

FIG. 8 is an isometric bottom view of the patient application assembly shown in FIG. 7;

FIG. 9 is a cross-sectional view of the patient application assembly shown in FIGS. 7 and 8;

FIG. 10 is a cross-sectional view of an example iontophoresis device in accordance with another aspect of the current disclosure; and

FIG. 11 is a cross-sectional view of a component of the iontophoresis device shown in FIG. 10.

### DETAILED DESCRIPTION

The present disclosure can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

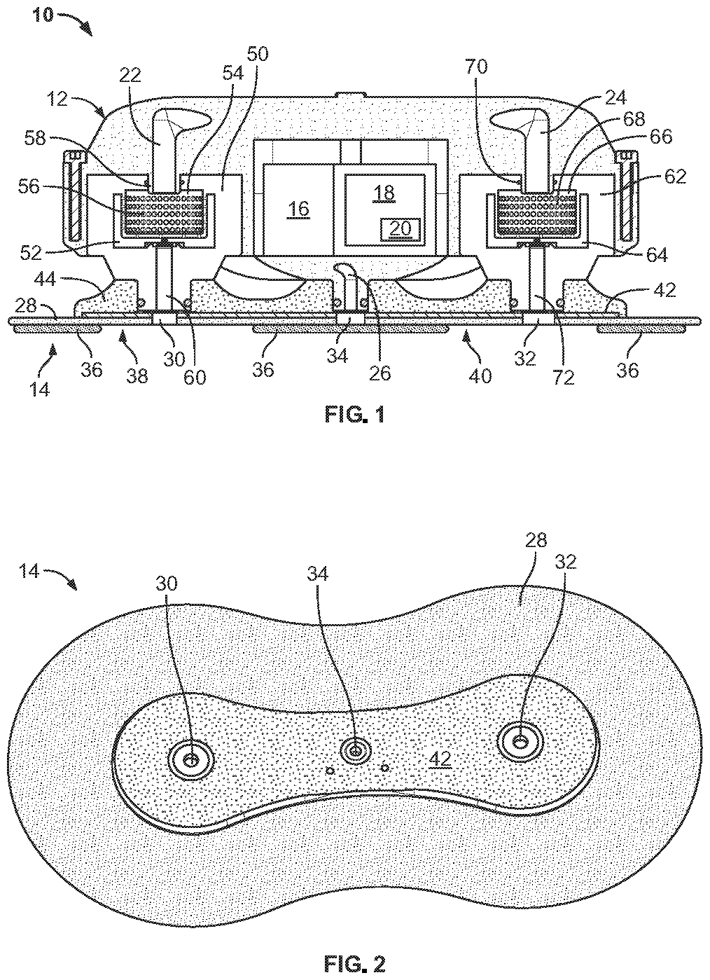

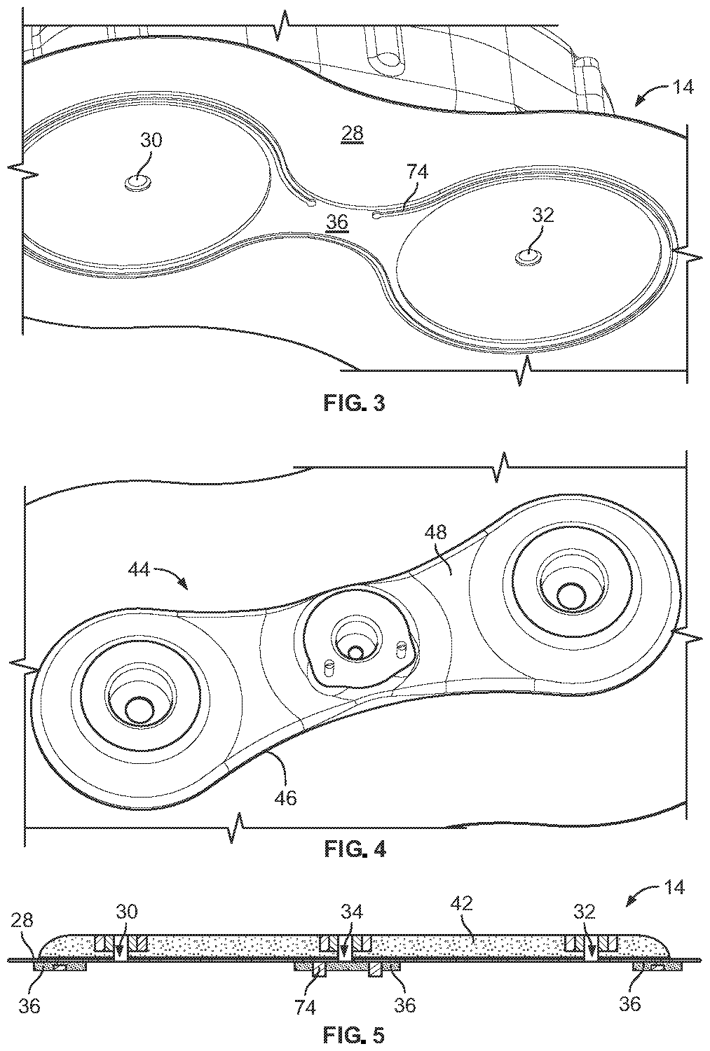

FIG. 1 is a cross-sectional illustration of an iontophoresis device 10 in accordance with one aspect of the present disclosure. The iontophoresis device 10 is configured for delivering a drug to a patient across a layer of skin (not shown). The iontophoresis device 10 is comprised of a removable/wireless/portable delivery apparatus 12. The terms removable/wireless/portable are intended to encompass any self-contained and powered delivery apparatus that is capable of operation without external controls. The removable delivery apparatus 12 is configured to removably engage a patient application assembly 14 as detailed in FIGS. 2-3 and 5. The removable delivery apparatus 12 includes a compressor 16 in communication with a processor 18 having logic controller 20 configured to control the compressor 16 to deliver pressurized air to a first and second air conduit 22, 24 as well as a vacuum to a vacuum conduit 26.

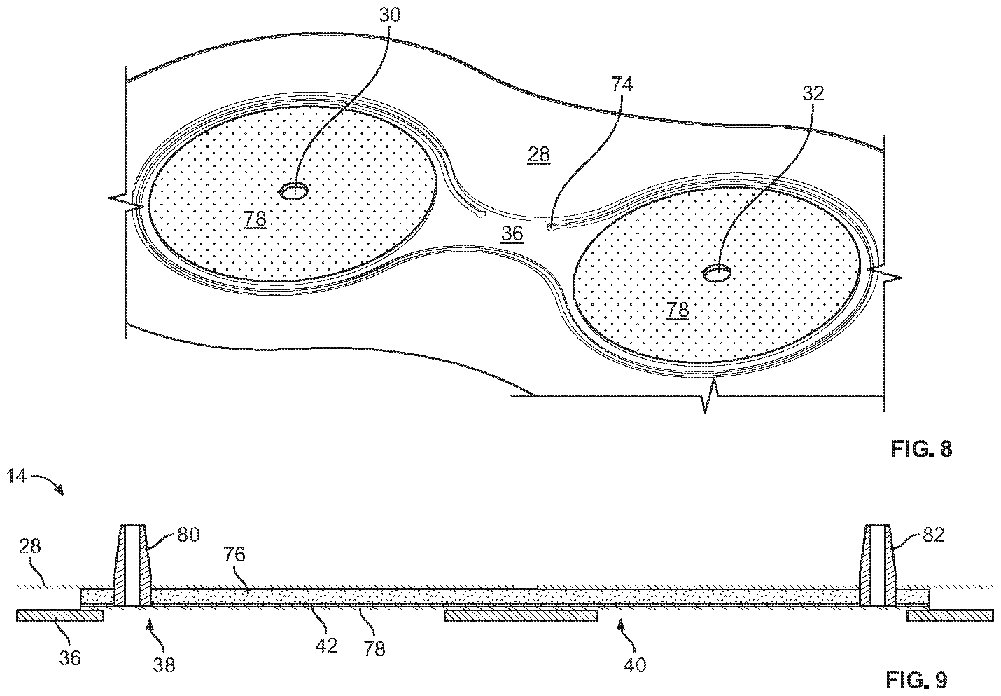

Referring now to FIGS. 2-3 and 5, which detail a patient application assembly 14 in accordance with one aspect of the current disclosure. The patient application assembly 14 may include a membrane 28 configured to be applied to the skin of a patient. In one aspect, the membrane 28 is contemplated to be comprised of Moleskin. However, any non-skin-reactant and non-permeable material may be utilized for the membrane 28. The membrane 28 defines a drug deliver aperture 30, a solvent delivery aperture 32 and a vacuum aperture 34. In this aspect, the patient application assembly 14 may further include a non-permeable frame 36 in communication with the membrane 28, the non-permeable frame 36 defining a drug receiving chamber 38 and a solvent receiving chamber 40. For the purposes of this disclosure, the term non-permeable shall refer to materials that do not allow for the passage of liquids or gasses there through. The term semi-permeable shall refer to materials that allow for the passage of gasses but not liquids there through. The term permeable shall refer to materials that allow for the passage of both liquids and gasses there trough. In at least one aspect of the disclosure, it is contemplated that the non-permeable frame 36 may be comprised of closed cell foam such as non-latex neoprene. The various layers of the patient application assembly 14 may be joined together by way of a medical device instant adhesive such as lactite. Although the non-permeable frame 36 may be formed in a variety of shapes, in one aspect, it is formed as a FIG. 8.

Additionally in this aspect, the patient application assembly 14 may further include a semi-permeable membrane 42 in communication with the drug delivery aperture 30, the solvent delivery aperture 32 and the vent aperture 34. The semi-permeable membrane 42 transferring vacuum from the vent aperture 34 to the drug delivery aperture 30 and to the solvent delivery aperture 32. In at least one aspect of the disclosure, the semi-permeable membrane 42 may comprise a hydrofera blue membrane. In this aspect, it is contemplated that the semi-permeable membrane 42 and the non-permeable frame 36 are mounted on opposing sides of the membrane 28. In this aspect, a non-permeable cover 44 includes a cover base 46 configured to surround and seal the semi-permeable membrane 42 and a cover top 48 configured to securely engage the removable delivery apparatus 12. The non-permeable cover 44 allows the vacuum from the vacuum conduit 26 to be distributed through the semi-permeable membrane 42 from the vent aperture 34 to the drug delivery aperture 30 and the solvent delivery aperture 32.

Referring back to FIG. 1, the removable delivery apparatus 12 may include a drug cartridge 50 defining a drug containing chamber 52 and a drug air chamber 54. The drug containing chamber 52 and the drug air chamber 54 are separated by a first flexible membrane 56. A first drug cartridge port 58 is in communication with one of the first air conduit 22 and the drug air chamber 54. A second drug cartridge port 60 is in communication with both the drug containing chamber 52 and the drug delivery aperture 30. When the compressor 16 is activated, pressurized air is introduced into the first air conduit 22 which flows through the first drug cartridge port 58 and thereby expands the first flexible membrane 56 into the drug containing chamber 52. This pressurizes any drugs within the drug containing chamber 52 and forces them through the second drug cartridge port 60, through the drug delivery aperture 30 and into the drug receiving chamber 38. It should be understood that the first drug cartridge port 58 and the second drug cartridge port 60 may be sealed with self-healing seals that only allow passage of air or liquid drugs when a sufficient pressure is achieved.

Similarly, in another aspect of the disclosure, the removable delivery apparatus 12 may include a solvent cartridge 62 defining a solvent containing chamber 64 and a solvent air chamber 66. The solvent containing chamber 64 and the solvent air chamber 66 are separated by a second flexible membrane 68. A first solvent cartridge port 70 is in communication with the second air conduit 24 and the solvent air chamber 66. A second solvent cartridge port 72 is in communication with both the solvent containing chamber 64 and the solvent delivery aperture 32. When the compressor 16 is activated, pressurized air is introduced into the second air conduit 24 which flows through the first solvent cartridge port 70 and thereby expands the second flexible membrane 68 into the solvent containing chamber 64. This pressurizes any solvent within the solvent containing chamber 66 and forces it through the second solvent cartridge port 72, through the solvent delivery aperture 32 and into the solvent receiving chamber 40. It should be understood that the first solvent cartridge port 70 and the second solvent cartridge port 72 maybe sealed with self-healing seals that only allow passage of air or liquid solvent when a sufficient pressure is achieved.

In one aspect of the disclosure, the logic controller 20 is configured to activate the compressor 16 to generate a vacuum in the vent aperture 34. The vacuum is transferred via the semi-permeable membrane 42 to the solvent receiving chamber 40 and the drug receiving chamber 38. Afterwards, the logic controller 20 is configured to add pressure to the first drug cartridge port 58 and the first solvent cartridge port 70. When the solvent has filled the solvent receiving chamber 40 and the drug has filled the drug receiving chamber 38, the logic controller 20 is configured to supply a current to an electrode 74 positioned on the non-permeable frame 38 and located between the non-permeable frame 38 and the patient's skin. The electrode 74 substantially surrounds the drug delivery aperture 30 and the solvent delivery aperture 32. Through the activation of current in the electrode 74, the iontophoresis process is activated and the drug and solvent are ionically drawn through the patient's skin.

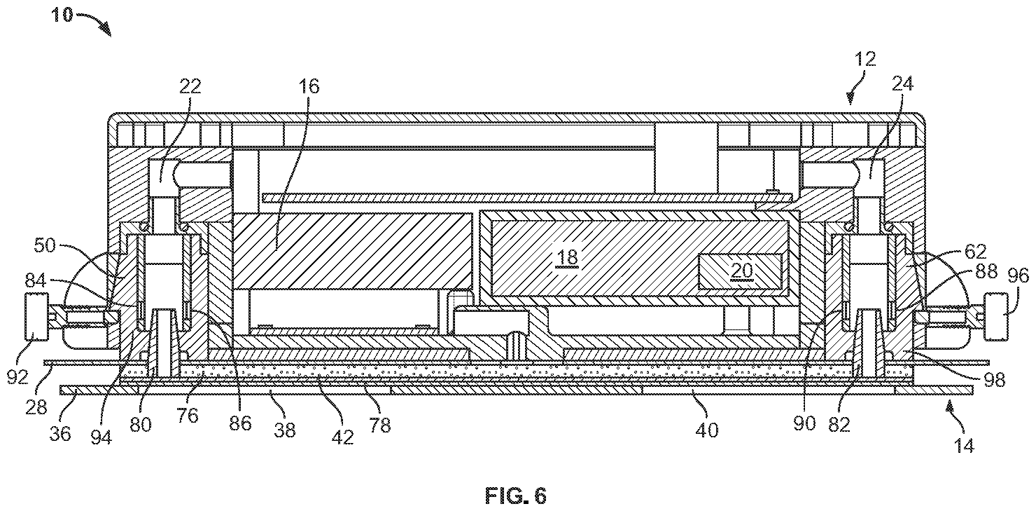

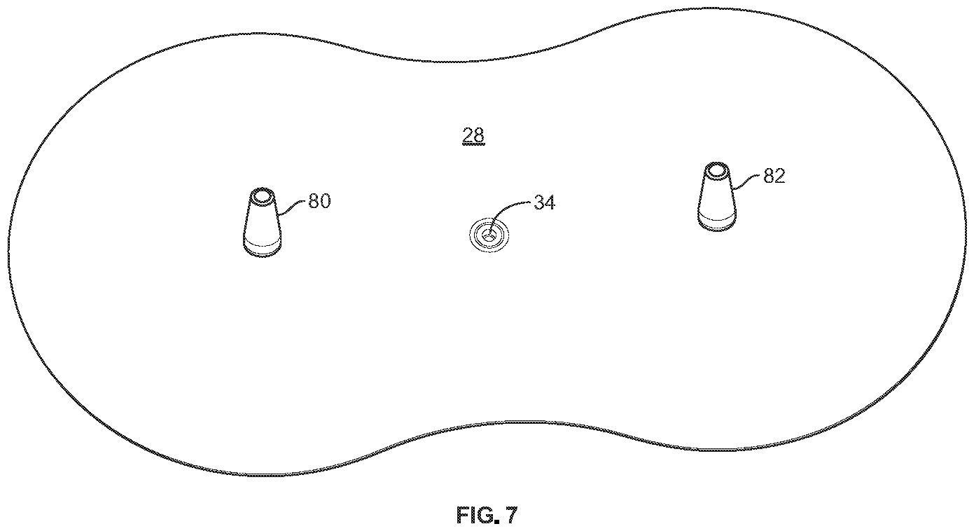

In another aspect of the disclosure, depicted in FIGS. 6-9, the iontophoresis device 10 may comprise the semi-permeable membrane 42 positioned between the non-permeable frame 36 and said membrane 28. In this aspect, the patient application assembly 14 may further include a bleeder cloth 76 positioned between the semi-permeable membrane 42 and the membrane 28. The bleeder cloth 76 is configured to distribute vacuum between the vent aperture 34 and the drug delivery aperture 30 and the solvent delivery aperture 32. In at least one aspect, the bleeder cloth 76 is contemplated to be polyester felt. In this aspect, the patient application assembly 14 may further include an electrode film layer 78 positioned between the semi-permeable membrane 42 and non-permeable frame 36. The electrode film layer 78 transfers current from the removable delivery apparatus 12 to the electrode 74.

In this aspect, the iontophoresis device 10 may include a conductive drug nozzle 80 in communication with the electrode film layer 78, the drug delivery aperture 32, and the drug receiving chamber 38. The conductive drug nozzle 80 transfers current from the removable delivery apparatus 12 to the electrode film layer 78. The removable delivery apparatus 12 further includes a conductive solvent nozzle 82 in communication with the electrode film layer 78, the solvent deliver aperture 34, and the solvent receiving chamber 40. The conductive solvent nozzle 82 transfers current from the electrode film layer 78 to the removable delivery apparatus 12. In this fashion, a conductive loop is formed through the electrode 74. In at least one aspect of the disclosure, the conductive drug nozzle 80 and the conductive solvent nozzle 82 are formed of a conductive material such as copper. The conductive drug nozzle 80 and the conductive solvent nozzle 82 act both as conduits for flow of drug and solvent as well electrical conduits for current to flow to the electrode 74.

In one aspect of the disclosure, it is contemplated that the drug cartridge 50 and the solvent cartridge 62 comprise removable and refillable cartridges. In this aspect the drug cartridge 50 may include a drug insertion port 84 and a drug relief port 86. The drug insertion port 84 and the drug relief port 86 comprise self-healing ports that allow a syringe to insert drugs into the drug insertion port 84 and another syringe to release pressure from the drug relief port 86 while the drugs are being inserted into the drug cartridge 50. Similarly, the solvent cartridge 62 may include a solvent insertion port 88 and a solvent relief port 90. These ports are self-healing ports as well that allow a syringe to insert solvent into the solvent insertion port 88 and another syringe to release pressure from the solvent relief port 90 while the solvent is being inserted into the solvent cartridge 62.

In still another aspect of the current disclosure, the removable delivery apparatus 12 may include a first conductive spring pin 92 engaging a first conductive portion 94 of the drug cartridge 50. The first conductive spring pin 92 retains the drug cartridge 50 within the removable delivery apparatus 12 and transfers current from said removable delivery apparatus 12 through the first conductive portion 94 to the conductive drug nozzle 80. Similarly, the removable delivery apparatus 12 may include a second conductive spring pin 96 engaging a second conductive portion 98 of the solvent cartridge 62. The second conductive spring pin 96 retains the solvent cartridge 62 within the removable delivery apparatus 12 and transfers current from the conductive solvent nozzle 82 through the second conductive portion 98 to the removable delivery apparatus 12.

The example embodiments described above use cartridges containing the agents in liquid form. However, significant advantage may be realized if the agent or agents (e.g., neostigmine and glycopyrrolate) are placed in the cartridges in a lyophilized (dry powder) form for administration because agents in dry powder form have significantly longer shelf life than agents in liquid form. This embodiment will, of course, require reconstitution of the lyophilized agent(s) with solvent (distilled water in this example) prior to delivery of agent(s) to the patch for administration. The function of the iontophoresis device is otherwise unchanged.

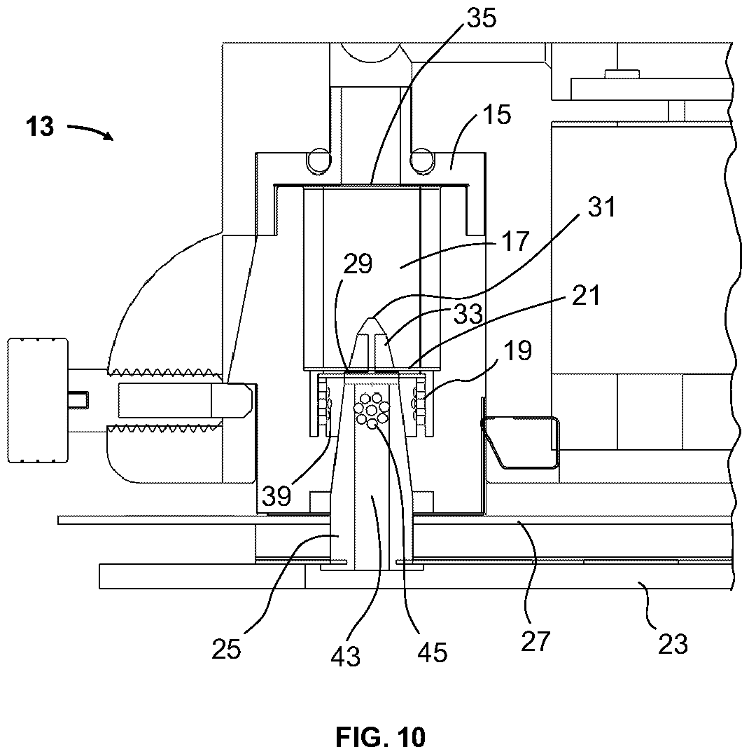

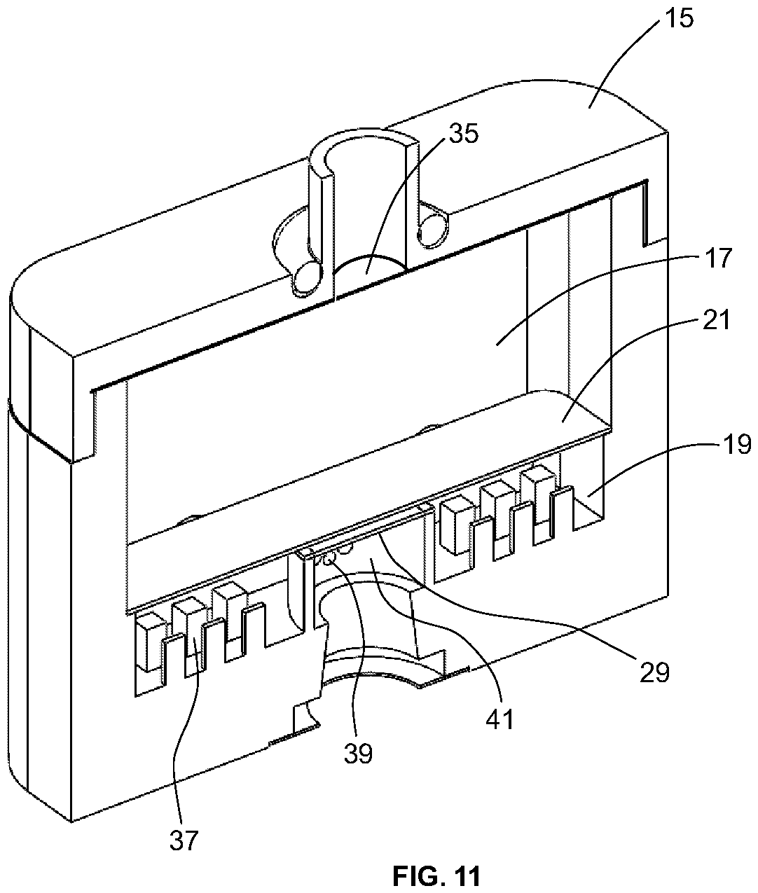

FIG. 10 shows an example embodiment of an iontophoresis device 13 which uses cartridges 15 containing lyophilized agents (see FIG. 11). In cartridge 15, the liquid solvent is stored in a solvent chamber 17 separate from a lyophilized agent chamber 19. The solvent chamber 17 is separated from the agent chamber 19 by an impermeable membrane, such as a thin foil separation membrane 21, until use.

When device 13 is ready for use, the membrane patch 23 (see FIG. 10) is placed on the patient, and the iontophoresis device 13 is placed onto barbs 25 projecting from the patch 23. As shown in FIG. 10, the barbs 25 pierce the patch's foil contact layer 27, then they pierce a self-sealing membrane 29 (see also FIG. 11), and finally the separation membrane 21. The purpose of the self-sealing membrane 29 is to seal around the barb 25 and force the liquid solvent to flow around it. The liquid solvent is directed to interact with the lyophilized agent in the agent chamber 19. The tip 31 of barb 25 is pointed to facilitate piercing of the various membranes and allow the liquid solvent from the solvent chamber 17 to flow into the lyophilized agent chamber 19 and mix with the agent or agents therein. One or more shaped cavities 33 may be arranged in barb 25 proximate to the barb tip 31. Shaped cavities 33 are designed to prevent the barb 25 from occluding the hole it pierces in the separation membrane 21 to enable free flow of the liquid solvent from the solvent chamber 17 into the lyophilized agent chamber 19.

Once the device 13 is installed on the patch 23 and initiated to function, the device compressor (not shown) pushes air against a flexible membrane 35 (for example, a nitrile membrane) positioned within and sealing the solvent chamber 17. The flexible membrane 35 expands into the solvent chamber 17, thereby displacing the liquid solvent into the lyophilized agent chamber 19. As the liquid solvent is forced into the lyophilized agent chamber 19 the liquid solvent mixes with the lyophilized agent. As shown in FIG. 11, turbulent flow extrusions 37 positioned within the agent chamber 19 create further turbulent flow of the liquid solvent, which serves to mix the lyophilized agent more uniformly into solution. The self-sealing membrane 29 forces the liquid solvent to flow through cartridge openings 39 in a barb isolation chamber 41, further mixing the lyophilized agent in the liquid solvent. The mixed agent in solution then enters a bore 43 in the barb 25 through intake openings 45. The solution then flows into the patch 23. Thus the design of the example embodiment of the iontophoresis device 13 ensures that the lyophilized agent and liquid solvent are kept separate to provide extended viability of the lyophilized agent(s) during storage.

Once the process of agent dispensing is initiated, the liquid solvent follows on a path to interact with the lyophilized agent with the extrusions 37, cartridge openings 39 and barb intake openings 45 that cause the liquid solvent to travel down a turbulent channel, thereby uniformly mixing the lyophilized agent(s) into solution for more predictable iontophoretic drug delivery.

Although the present disclosure contemplates the application is directly applicable to the self-administration of a wide variety of drugs, in one particular aspect the disclosure contemplates the application of a combination of neostigmine (NEO; a cholinergic agent) and glycopyrrolate (GLY; a selective cardiopulmonary anticholinergic agent) to facilitate bowel moments in individuals. This allows self-administration of such a motility drug in the privacy of an individual's home as well as self-control over their bodily functions.

1. An iontophoresis device (10) for delivering a drug to a patient across a layer of skin, said iontophoresis device (10) characterized by:
a membrane (28) applied to said skin, said membrane (28) having a drug delivery aperture (30), a solvent delivery aperture (32) and a vent aperture (34) for applying a vacuum between said membrane (28) and said skin;
a drug cartridge defining a drug containing chamber and a drug air chamber, said drug containing chamber and said drug air chamber separated by a first flexible membrane, said drug containing chamber in fluid communication with said drug delivery aperture (30), said drug cartridge having a first drug cartridge port in fluid communication with said drug air chamber for pressurizing said drug containing chamber and a second drug cartridge port in fluid communication with said drug delivery aperture (30);
a solvent cartridge defining a solvent containing chamber and a solvent air chamber, said solvent containing chamber and said solvent air chamber separated by a second flexible membrane, said solvent containing chamber in fluid communication with said solvent vehicle aperture (32), said solvent cartridge having a first solvent port in fluid communication with said solvent air chamber for pressurizing said solvent containing chamber and a second solvent port in fluid communication with said solvent delivery aperture (32);
an electrode extending between a positive terminal and a negative terminal, said electrode being positioned between said membrane and said skin, said electrode substantially surrounding said drug delivery aperture (30) and said solvent delivery aperture (32).

2. The iontophoresis device (10) of claim 1, further characterized by:
a removable delivery apparatus (12) including a compressor (16) in communication with a processor (18) configured with logic controller (20) to control said compressor (16) to deliver a vacuum to said vent aperture (34) and a pressure to said first drug cartridge port and said first solvent cartridge port, said logic controller (20) further configured to supply current to said electrode:
wherein said drug cartridge and said solvent cartridge are housed within said removable delivery apparatus (12).

3. The iontophoresis device (10) of claim 1, further characterized by:
a non-permeable frame (36) in communication with said membrane (12), said non-permeable frame (36) defining a drug receiving chamber (38) and a solvent receiving chamber (40), said drug receiving chamber (38) in communication with said drug delivery aperture (30) and said solvent receiving chamber (40) in communication with said solvent receiving aperture (32), said electrode positioned between said non-permeable frame and said skin.

4. The iontophoresis device (10) of claim 1, further characterized by:
a semi-permeable membrane (42) in communication with said drug delivery aperture (30), said solvent delivery aperture (32) and said vent aperture (34), said semi-permeable membrane (42) transferring vacuum from said vent aperture (34) to said drug delivery aperture (30) and to said solvent delivery aperture (32).

5. The iontophoresis device (10) of claim 4, wherein said non-permeable frame (36) and said semi-permeable membrane (42) are positioned on opposing surfaces of said membrane (28), and wherein the iontophoresis device (10) is further characterized by:
a non-permeable cover (44) configured to surround said semi-permeable membrane (42), said non-permeable cover (44) configured to engage said removable delivery apparatus.

6. The iontophoresis device (10) of claim 4, wherein said semi-permeable membrane (42) is positioned between said non-permeable frame (36) and said membrane (28); and
wherein the iontophoresis device (10) is further characterized by:
a bleeder cloth (76) positioned between said semi-permeable membrane (42) and said membrane (28), said bleeder cloth (76) configured to distribute vacuum between said vent aperture (34) and said drug delivery aperture (30) and said solvent delivery aperture (32).

7. The iontophoresis device (10) of claim 6, further characterized by:
an electrode film layer (78) positioned between said semi-permeable membrane (42) and non-permeable frame (36), said electrode film layer (78) transferring current from said removable delivery apparatus (12) to said electrode (74).

8. The iontophoresis device (10) of claim 7, further characterized by:
a conductive drug nozzle (80) in communication with said electrode film layer (78), said drug delivery aperture (30), and said drug receiving chamber (38), said conductive drug nozzle (80) transferring current from said removable delivery apparatus (12) to said electrode film layer (78); and
a conductive solvent nozzle (82) in communication with said electrode film layer (78), said solvent deliver aperture (32), and said solvent receiving chamber (40), said conductive solvent nozzle (82) transferring current from said electrode film layer (78) to said removable delivery apparatus (12).

9. The iontophoresis device (10) of claim 1; wherein said drug cartridge (50) and said solvent cartridge (62) comprise removable and refillable cartridges.

10. The iontophoresis device (10) of claim 9, further characterized by:
a first conductive spring pin (92) engaging a first conductive portion (94) of said drug cartridge (50), said first conductive spring pin (92) retaining said drug cartridge (50) within said removable delivery apparatus (12) and transferring current from said removable delivery apparatus (12) through said first conductive portion (94) to said conductive drug nozzle (80); and
a second conductive spring pin (96) engaging a second conductive portion (98) of said solvent cartridge (62), said second conductive spring pin (96) retaining said solvent cartridge (62) within said removable delivery apparatus (12) and transferring current from said conductive solvent nozzle (82) through said second conductive portion (98) to said removable delivery apparatus (12).

11. A method of delivering a drug to a patient across a layer of skin using an iontophoresis device (10), said method comprising:
applying a membrane (28) to said skin, said membrane (28) having a drug delivery aperture (30), a solvent delivery aperture (32) and a vent aperture (34) for applying a vacuum between said membrane (28) and said skin creating a partial vacuum between said membrane (28) and said skin:
placing a drug cartridge (50) in communication with said drug delivery aperture (30), said drug cartridge (50) defining a drug containing chamber (52) and a drug air chamber (54), said drug containing chamber (52) and said drug air chamber (54) separated by a first flexible membrane (56), said drug containing chamber (52) in fluid communication with said drug delivery aperture (30), said drug cartridge (50) having a first drug cartridge port (58) in fluid communication with said drug air chamber (54) for pressurizing said drug containing chamber (52) and a second drug cartridge port (60) in fluid communication with said drug delivery aperture (30);
placing a solvent cartridge (62) in communication with said solvent delivery aperture (32), said solvent cartridge (62) defining a solvent containing chamber (64) and a solvent air chamber (66), said solvent containing chamber (64) and said solvent air chamber (66) separated by a second flexible membrane (68), said solvent containing chamber (64) in fluid communication with said solvent vehicle aperture (32), said solvent cartridge (62) having a first solvent port (70) in fluid communication with said solvent air chamber (66) for pressurizing said solvent containing chamber (64) and a second solvent port (72) in fluid communication with said solvent delivery aperture (32):
pressurizing said drug air chamber (54) forcing said first flexible member (56) to pressurize said drug containing chamber (52), said drug containing chamber (52) being in fluid communication with said skin through said drug delivery aperture (30), a drug flowing from said drug containing chamber (52) to said skin adjacent to said electrode (74);
pressurizing said solvent air chamber (66) forcing said second flexible member (68) to pressurize said solvent containing chamber (64), said solvent containing chamber (64) being in fluid communication with said skin through said solvent delivery aperture (32), a solvent flowing from said solvent containing chamber (64) to said skin adjacent to said electrode (74); and
applying an electric field to said electrode (74) to effect iontophoresis.

12. (canceled)

13. (canceled)

14. (canceled)

15. (canceled)

16. (canceled)

17. (canceled)

18. (canceled)

19. (canceled)

20. (canceled)

21. An iontophoresis device (13) for delivering a drug to a patient across a layer of skin, said iontophoresis device (13) characterized by:
a membrane (23) applied to said skin, said membrane (23) having a barb (25) projecting therefrom and defining a bore (43) providing fluid communication through said membrane (23) to said skin, intake openings (45) in said barb providing fluid communication with said bore (43), and a vent aperture for applying a vacuum between said membrane (23) and said skin;
a drug cartridge (15) defining a solvent chamber (17) and an agent chamber (19) adapted to contain a lyophilized agent, said solvent chamber (17) and said agent chamber (19) separated by an impermeable membrane (21), said agent chamber (19) being in fluid communication with said bore (43) through said intake openings (45), said solvent chamber (17) being in fluid communication with a compressor for pressurizing said solvent chamber (17);
a barb isolation chamber (41) positioned within said agent chamber (19), said barb isolation chamber (41) adapted to receive said barb (25), a self-sealing membrane (29) isolating said barb isolation chamber (41) from said agent chamber (19), a plurality of cartridge openings (39) in said barb isolation chamber (41) providing fluid communication between said agent chamber (19) and said barb isolation chamber (41); wherein
when said barb (25) is received within said barb isolation chamber (41) said barb (25) pierces said self-sealing membrane (29) and said impermeable membrane (21) thereby releasing solvent from said solvent chamber (17) into said agent chamber (19), said solvent mixing with said agent and entering said bore (43) through said cartridge openings (39) and said intake openings (45) for application to said skin.

22. The iontophoresis device (13) of claim 21, further characterized by:
a removable delivery apparatus (12) including a compressor (16) in communication with a processor (18) configured with logic controller (20) to control said compressor (16) to deliver a vacuum to said vent aperture and a pressure to said solvent chamber (17).

23. The iontophoresis device (13) of claim 21, further characterized by a plurality of turbulent flow extrusions (37) positioned within said agent chamber (19), said turbulent flow extrusions (37) adapted to create turbulent flow of said solvent and thereby promote mixing of said solvent and said agent.

24. The iontophoresis device (13) of claim 21, further characterized by said self-sealing membrane (29) sealing around said barb (25) to force solvent to flow through said cartridge openings (39) when said barb (25) pierces said self-sealing membrane (29).

25. The iontophoresis device (13) of claim 21, wherein said barb (25) comprises a pointed tip (31).

26. The iontophoresis device (13) of claim 25, wherein said barb 25 comprises a plurality of shaped cavities (33) arranged proximate to said barb tip (31), said shaped cavities (33) adapted to allow free flow of said solvent between said solvent chamber (17) and said agent chamber (19) when said barb pierces said impermeable membrane (21).

27. The iontophoresis device (13) of claim 21, further characterized by said impermeable membrane (21) comprising a thin foil separation membrane.

28. The iontophoresis device (13) of claim 21, further characterized by a flexible membrane positioned within said solvent chamber (17).

29. The iontophoresis device (13) of claim 21, further characterized by said cartridge (15) comprising a solvent in said solvent chamber (17) and an agent in said agent chamber (19).

30. The iontophoresis device (13) of claim 29, further characterized by said solvent comprising distilled water and said agent comprising neostigmine and glycopyrrolate.
